(19) United States
(12) Patent Application Publication
(10) Pub. No.: US 2024/0150263 A1
(43) Pub. Date: May 9, 2024

(54) Process and System for Producing a Target Compound

(71) Applicants: LINDE GMBH, Pullach (DE); Clariant International Ltd., Muttenz (CH)

(72) Inventors: Mathieu Zellhuber, Martinsried (DE); Martin Schubert, München (DE); Andreas Meiswinkel, Rimsting (DE); Gerhard Mestl, Bruckmühl (DE); Klaus Wanninger, Bruckmühl (DE); Peter Scheck, München (DE); Anina Wohl, München (DE)

(21) Appl. No.: 18/549,582

(22) PCT Filed: Mar. 14, 2022
(86) PCT No.: PCT/EP2022/056570

§ 371 Date: Sep. 7, 2023

(30) Foreign Application Priority Data
Mar. 15, 2021 (DE) 102021202495.5

(51) Int. Cl. C07C 5/48; B01J 8/06; B01J 23/22; B01J 23/28; B01J 27/057; B01J 35/50; B01J 37/00; B01J 37/02; B01J 37/03; B01J 37/10

(52) U.S. Cl. CPC C07C 5/48; B01J 8/06; B01J 23/22; B01J 23/28; B01J 27/0576; B01J 35/50; B01J 37/009; B01J 37/0236; B01J 37/031; B01J 37/10; B01J 2208/021; B01J 2208/025; C07C 2523/22; C07C 2523/28; C07C 2527/057

(57) ABSTRACT

A process for producing a target compound includes forming a feed mixture containing at least one reactant compound. The feed mixture is distributed to parallel reaction tubes of one or more shell-and-tube reactors and subjected to oxidative catalytic conversion in the reaction tubes. Steam is added to the feed mixture in an amount such that a steam fraction of the feed mixture is 5 to 95 vol %, oxygen is added to the feed mixture in the form of a fluid containing at least 95 vol % oxygen, and the oxidative catalytic conversion is carried out using one or more catalysts containing the metals molybdenum, vanadium, niobium and optionally tellurium.

### CROSS-REFERENCE TO RELATED APPLICATIONS

This application is the national phase of, and claims priority to, International Application No. PCT/EP2022/056570, filed Mar. 14, 2022, which claims priority to German Patent Application No. DE102021202495.5, filed Mar. 15, 2021.

### FIELD OF THE INVENTION

The invention relates to a method and apparatus for producing a target compound.

### BACKGROUND

The oxidative dehydrogenation (ODH) of kerosenes with two to four carbon atoms is known in principle. In ODH, said kerosenes are reacted with oxygen to form, among other things, the respective olefins and water. In particular, the invention relates to the oxidative dehydrogenation of ethane to ethylene, hereinafter also referred to as ODHE. However, the invention is in principle not limited to the oxidative dehydrogenation of ethane, but may also extend to the oxidative dehydrogenation (ODH) of other kerosenes such as propane or butane. In this case, the following explanations apply accordingly.

ODH(E) can be advantageous over more established olefin production processes such as steam cracking or catalytic dehydrogenation. For example, there is no thermodynamic equilibrium limitation due to the exothermic nature of the reactions involved and the virtually irreversible formation of water. ODH(E) can be carried out at comparatively low reaction temperatures. In principle, no regeneration of the catalysts used is required, since the presence of oxygen enables or causes in situ regeneration. Finally, in contrast to steam cracking, smaller amounts of worthless by-products such as coke are formed.

For further details regarding ODH(E), reference should be made to relevant literature, for example, Ivars, F. and López Nieto, J. M., Light Alkanes Oxidation: Targets Reached and Current Challenges, in Duprez, D. and Cavani, F. (eds.), Handbook of Advanced Methods and Processes in Oxidation Catalysis: From Laboratory to Industry, London 2014: Imperial College Press, pages 767-834, or Gartner, C. A. et al, Oxidative Dehydrogenation of Ethane: Common Principles and Mechanistic Aspects, ChemCatChem, vol. 5, no. 11, 2013, pages 3196 to 3217, and X. Li, E. Iglesia, Kinetics and Mechanism of Ethane Oxidation to Acetic Acid on Catalysts Based on Mo—V—Nb Oxides, J. Phys. Chem. C, 2008, 112, 15001-15008, referenced.

WO 2019/243480 A1 proposes a process for producing one or more olefins and one or more carboxylic acids by subjecting one or more kerosenes to oxidative dehydrogenation. A reactor having a plurality of reaction zones is used for the oxidative dehydrogenation, a gas mixture comprising the one or more kerosenes is passed sequentially through the reaction zones, and at least two of the plurality of reaction zones are subjected to temperature manipulation to varying degrees.

In particular, MoVNb-based catalyst systems have shown promise for ODH(E), as mentioned for example in F. Cavani et al, “Oxidative dehydrogenation of ethane and propane: How far from commercial implementation?”, Catal. Today, 2007, 127, 113-131, mentioned. Additional Te-containing catalyst systems can also be used. Where reference is made herein to a “MoVNb-based catalyst system” or a “MoVTeNb-based catalyst system,” let this be understood to mean a catalyst system comprising the elements mentioned as a mixed oxide, also expressed as MoVNbOₓ and MoVTeNbOₓ, respectively. The indication of Te in brackets stands for its optional presence. The invention is used in particular with such catalyst systems.

In the case of ODH, significant amounts of the respective carboxylic acids of the kerosenes used, in particular acetic acid in the case of ODHE, are formed as by-products under industrially relevant reaction conditions, especially when catalysts based on MoVNb(Te)Oₓ are used. For economical plant operation, therefore, a co-production of olefins and the carboxylic acids is generally unavoidable when using the catalyst type described, but a preferential formation of olefins is desirable.

According to the state of the art, conversion and selectivity in ODH(E) are mostly limited and only for a few catalyst systems, such as in particular the mentioned MoVNbTeOₓ systems, sufficiently high conversions and selectivities have recently been reported, which promise an economically reasonable technical implementation that can be competitive to e.g. steamcracking. Nevertheless, no such technical implementation has been realized so far and the state of the art is limited to laboratory- or at most pilot-scale plants.

In general, low-oxygen conditions are used in technically relevant oxidative processes such as the production of maleic anhydride (MSA) from butane, butene or benzene or the two-step synthesis of acrylic acid from propylene via the intermediate acrolein. Oxygen-depleted air is usually used as oxidant. An example is DE 198 37 519 A1 for the oxidation of propane to acrolein and/or acrylic acid. An up-to-date overview of MSA synthesis, which is carried out without exception using air as oxidant, can also be found, for example, in P. V. Mangili et al, “Eco-efficiency and techno-economic analysis for maleic anhydride manufacturing processes,” Clean Technol. Environ. Policy 2019, 21, 1073-1090.

In principle, however, the use of air or oxygen-enriched air as an oxidant is also discussed in the context of process intensification. Last but not least, the avoidance of explosive mixtures always plays a role in this context. For this reason, too, air is preferably used as an oxidant.

The additional feed of nitrogen or another dilution medium in addition to air also leads in the result to comparable conditions as with oxygen-depleted air. The introduction of nitrogen with air or oxygen enriched or depleted air then requires nitrogen separation in product purification and separation. Although such a nitrogen separation is technically feasible without any problems, especially for heavy oxygenates or hydrocarbons, it is often difficult or costly to implement due to the low boiling point of nitrogen, especially in the case of ODH(E). The lower the boiling point of the desired target product (ethylene in the case of ODHE), the greater the effort required. In contrast to MSA and acrylic acid, ethylene can only be condensed out under cryogenic conditions.

EP 2 716 621 A1, EP 2 716 622 A1, WO 2018/115416A1, WO 2018/115418 A1, WO 2018/082945 A1 and EP 3 339 277 A1 of the applicant disclose the supply of pure oxygen, e.g. obtained from a distillative air separation, as an alternative option in addition to the use of air or oxygen-enriched or oxygen-depleted air, but do not disclose the associated special requirements for reaction control and, in particular, the necessary coordination of catalyst and reaction control. WO 2020/074750 A1 also mentions the use of oxygen or oxygen-enriched air as an oxidant, but likewise does not go into further detail on the associated challenges in technical implementation. On the other hand, the provision of oxygen by suitable processes such as distillative air separation or pressure swing adsorption is, as already stated in the applications cited above, an established technology that can be implemented easily and economically favorably on almost any scale.

According to the state of the art, strongly exothermic reactions such as ODH(E) are preferably carried out in fixed-bed reactors, in particular in cooled shell-and-tube reactors. The coolant is fed in co-current or counter-current to the direction of flow of the reaction inlet stream, advantageously in counter-current, since here the dissipated heat from the later reaction zones can be used in the front reaction zones. Depending on the temperature range of the reaction, thermal oils or, in particular, molten salts are used as coolants. For corresponding reactions, including ODH(E), the use of a reactor bed with several zones is generally known. Basic principles are described, for example, in WO 2019/243480 A1 of the applicant. This document discloses the principle that different catalyst beds or corresponding reaction zones, which have different catalyst loadings and/or catalyst activities per unit space, are used.

In general, strongly exothermic reactions such as those of interest here, e.g. oxidative reactions such as ODH, in particular ODHE, require effective temperature control and dissipation of the resulting heat of reaction due to the exothermic nature of the reaction. This temperature control is all the more important the higher the desired conversion, since the amount of heat released increases proportionally with increasing conversion.

Frequently, in addition to the target product (in the case of ODH an olefin, in the case of ODHE ethylene), further oxygen-containing species are formed in corresponding reactions, such as carbonyl compounds, carboxylic acids and/or carbon oxides, i.e. carbon monoxide and/or carbon dioxide. At excessively high temperatures or local temperature peaks, which can occur or be caused in particular by temperature gradients, the undesired formation of the carbon oxides in particular is promoted. Here, therefore, high temperatures and/or local temperature peaks, which as described are accompanied by high conversion, again have a strong negative effect.

Therefore, one of the key challenges is to control the exothermic reaction even at commercially preferred high conversions (e.g., greater than 40%, 50%, 60%, 70%, 80%, 85%, or 90% per pass) and high reaction rates. For ODHE in an embodiment according to the invention, typically only around 40%, 50%, 60% or 66% conversion is achieved using concentrated feed streams and using pure oxygen, as the maximum conversion is limited by heat removal and temperature rise. Higher conversions then lead to a significant loss of selectivity, which generally no longer permits economical operation of the process.

As described, in other cases, in addition to appropriate cooling of the reaction volume, dilution of the reaction gas typically occurs. For example, higher conversions (of more than 70%, 80%, 85% or 90%) in MSA synthesis are achieved only with appropriate dilution and/or use of air or oxygen-depleted air. However, the measures mentioned can only be implemented in practice, especially in ODH(E), to a certain extent, as this increases both the equipment requirements and the associated capital expenditure for such a process (larger reactors/vessels/apparatus) and also significantly increases the energy required for separation and compression steps, which not only worsens the life cycle assessment (carbon dioxide footprint) but also increases the running costs of a process. As described, the separation of inert dilution media (nitrogen, argon, carbon dioxide . . . ) from low-boiling components such as ethylene is a particular challenge here.

The invention sets out to demonstrate improved and more effective ways of producing target compounds of the type mentioned.

### SUMMARY

According to one embodiment, a method for producing a target compound includes forming a feed mixture containing at least one reactant compound; distributing the feed mixture to parallel reaction tubes of one or more shell-and-tube reactors; and subjecting the feed mixture to an oxidative catalytic reaction in the reaction tubes. Ethane is used as reactant compound and the oxidative catalytic process is carried out as oxidative dehydrogenation of the ethane. Steam is added to the feed mixture in an amount such that a steam content of the feed mixture is from 5 to 95% by volume. Oxygen is added to the feed mixture in the form of a fluid containing at least 95% by volume of oxygen. The oxidative catalytic reaction is carried out using one or more catalysts containing the metals molybdenum, vanadium, and niobium which is or are arranged in a plurality of reaction zones of the one or more shell-and-tube reactors, wherein the one or the plurality of catalysts in the reaction zones is or are provided with a different catalyst loading and/or a different catalyst activity per unit space.

According to another embodiment, a plant for producing a target compound includes one or more tube shell-and-tube reactors having reaction tubes arranged in parallel The one or more reactors is or are adapted to form a feed mixture (A) containing at least one reactant compound; distribute the feed mixture to reaction tubes (10) of the tube bundle reactor(s); and subject the feed mixture to an oxidative catalytic reaction in the reaction tubes. The plant is adapted to add steam to the feed mixture in an amount such that a steam fraction of the feed mixture is 5 to 95% by volume. Oxygen is added to the feed mixture in the form of a fluid containing at least 95% by volume oxygen. One or more catalysts containing the metals molybdenum, vanadium, and niobium are used for the oxidative catalytic reaction in the reaction tubes.

### BRIEF DESCRIPTION OF THE DRAWINGS

FIG. 1A illustrates different catalyst activities for differently prepared catalysts for partial use in the invention.

FIG. 1B illustrates temperature profiles for differently prepared catalysts for partial use in the invention.

FIG. 2 illustrates long-term stability data.

FIG. 3 illustrates a plant according to one embodiment of the invention in simplified schematic view.

FIG. 4 illustrates a reactor according to one embodiment of the invention in simplified schematic view.

### WRITTEN DESCRIPTION

In contrast to the above-mentioned established oxidation processes, the process according to the invention is based on the use of pure oxygen as oxidant. This pure oxygen can be easily and inexpensively provided from suitable sources, such as distillative air separation units or even pressure swing adsorption. One aspect of the invention is to match catalyst and process in an optimum manner and to achieve particular advantages in this connection.

Herein, “pure” oxygen is to be understood to include mixtures with an oxygen content of at least 95%, 98%, 99%, 99.5% or 99.9%; in particular, it may be so-called “technical” oxygen.

In addition, a suitable dilution medium must be added to the process. In the above-mentioned processes, this task is usually performed at least proportionally by nitrogen. In principle, other inert dilution media, in particular gaseous ones, can also be used, whereby an “inert dilution medium” is to be understood here as one or a mixture of several components which are not reacted or are reacted only to an insignificant extent in the oxidative dehydrogenation, in particular argon, helium, carbon dioxide or water vapor. In the context of the invention, on the other hand, in accordance with WO 2018/115416 A1, WO 2018/115418 A1 and WO 2019/243480 A1, water vapor is used with particular preference, which can not only be separated particularly easily and efficiently by condensation, but at the same time enables advantageous selectivity control and moderation of the catalyst selectivity. Furthermore, due to its high heat capacity, water vapor in particular enables improved distribution of the reaction heat over the reactor tube.

Such conditions, which are very advantageous for a commercial process, have not yet been implemented or described for an industrially relevant process setup for an oxidative conversion of hydrocarbons.

In other words, the invention proposes a process for the preparation of a target compound, wherein a feed mixture containing at least one reactant compound is formed, distributed to parallel reaction tubes of one or more shell-and-tube reactors, and subjected to oxidative catalytic conversion in the reaction tubes.

According to the invention, water vapor is added to the feed mixture in an amount such that a water vapor fraction of the feed mixture is 5 to 95% by volume, oxygen is further added to the feed mixture in the form of a fluid containing at least 95% by volume oxygen, and the oxidative catalytic conversion is carried out using one or more catalysts containing the metals molybdenum, vanadium, niobium and optionally tellurium. As explained in detail previously, such catalysts are particularly advantageous for corresponding processes.

According to the invention, the one or more catalysts are provided in reaction zones of the one or more shell-and-tube reactors, which are arranged one behind the other in a flow direction and through which flow takes place in the flow direction. The reaction zones are each formed by correspondingly formed sections of the reaction tubes. The one or the plurality of catalysts is or are provided in the plurality of reaction zones with a different catalyst loading and/or a different catalyst activity per unit space, thereby achieving an activity gradation between the reaction zones. Optionally, the reaction zones can also be heated differently.

At least one reaction zone arranged downstream in the direction of flow is thereby formed in particular with a higher catalyst loading and/or with a higher catalyst activity per unit space than in a reaction zone arranged upstream thereof in the direction of flow.

Particularly advantageous is the guidance of the reaction in several reaction zones provided according to the invention, which can be formed by catalyst layers of different activity gradations (in particular increasing activity in the flow direction of the reaction feed stream) and/or differently heated zones (cf. WO 2019/243480 A1). This results in a “stretching” of the temperature hotspot over the length of the reactor or a distribution of the temperature hotspot to several local hotspots over the length of the reactor, whereby the relative height of the respective hotspots to the coolant temperature (1st hotspot) or to the final temperature of the preceding reaction zone (hotspots of the following zones) is always significantly smaller than the temperature difference from the coolant temperature to the global hotspot of an insert catalyst bed or of only one reaction zone. This has the advantages explained further below.

The combination of the process parameters proposed in accordance with the invention—also including a coordinated reactor design as described below and the aforementioned activity gradation between the reaction zones—with a catalyst based on MoVNbOₓ or, in particular, MoVNbTeO used in accordance with the invention enables a high conversion with particularly high selectivity and the lowest possible inert content (here, in particular, water vapor, nitrogen or other inert gaseous dilution media can be completely avoided) in the feed stream to the desired value products, in particular ethylene and acetic acid.

The advantages according to the invention are achieved in particular by a combination of three measures comprising (1) the process conditions mentioned and further explained below, (2) a provision of the catalyst in several zones, and (3) in particular a catalyst formulation mentioned below. An increase in selectivity towards ethylene is possible, as also explained in the examples, by maintaining a higher minimum temperature as well as a higher average temperature in the catalyst bed of the multiple reaction zones. This in turn enables outstanding yields and particularly economical operation of the process. Energy input and carbon dioxide emissions are minimized.

By dispensing with low-boiling diluents, a significant reduction in the separation effort in the separation and product purification section can be achieved. An increase in operational safety results from a lower risk of thermal runaway of the reaction, as also explained in connection with the examples.

The invention leads to stable reactor and catalyst performance over a long period of time, as evidenced by relevant experiments.

In particular, the invention can be used with specific hourly gas or weight space velocities (GHSV, Gas Hourly Space Velocity; WHSV, Weight Hourly Space Velocity). The GHSV can be in particular 400 to 10 000 (Nm³/h)_{Gas}/m³_{catalyst} or h⁻¹ and the WHSV in particular 0.8 to 25 (kg/h)_{Gas}/kg_{catalyst active mass} or h⁻¹, where Nm³ denote standard cubic meters. The GHSV is determined in particular at standard temperature (0° C.) and pressure (1 bar abs.) and, as can be taken from the above assumption, refers to a catalyst volume, whereas the WHSV refers to the mass of the active catalyst.

The invention may comprise separating the unreacted portion of the reactant compound, in particular a kerosene such as ethane in the ODH(E) in a disassembly section and returning it at least partially to the reactor(s).

In principle, the execution of the process according to the invention also appears to be suitable for other oxidation process, in particular the oxidation of propane/propylene to acrylic acid is interesting and offers potential. The invention is therefore expressly not limited to ODH(E).

Advantageously, the water vapor content of the feed mixture used in accordance with the invention is 10 to 50% by volume, in particular 14 to 35% by volume. Surprisingly, it has been shown that at such water vapor contents, on the one hand, stable catalyst operation can be ensured (a certain lower limit for the water content in the feed stream is advantageous for stable catalyst operation, as already shown in EP 3 558 910 B1) and, on the other hand, the efficiency of the process can also be ensured. For the latter aspect, this concerns in particular the upper limit of the water vapor fraction given here. Water vapor simultaneously serves as an inert medium or also as a moderator, but an excessively large water vapor fraction reduces the efficiency and thus also the economy of the process. In particular, in the context of the invention, steam can be used as essentially the only dilution medium. Thus, in particular, no other inert gas is used in appreciable proportions. With respect to efficiency, essential elements are the energy requirement for steam generation and the dimensioning of apparatus, in particular the steam generator, the reactor, downstream heat exchangers and separators. In the shell-and-tube reactor of the invention in particular, the aim is to achieve the highest possible space-time yield of valuable product. By optimizing the water content accordingly, it is also possible to achieve the highest possible concentration of acetic acid in the condensate. This also minimizes the equipment and energy required for further concentration of acetic acid as a valuable product.

The feed mixture is formed in particular in such a way that a ratio of a proportion of oxygen in the feed mixture to a proportion of the at least one starting compound in the feed mixture is at least 0.20, 0.25, 0.30 or 0.35 and up to 0.5 or 1.0. At such oxygen contents, a very high overall selectivity to the preferred value products ethylene and acetic acid can be achieved in particular at a targeted ethane conversion in the range of 40 to 60% and without the addition of further dilution medium than the previously mentioned steam. In particular, therefore, the proportions of the undesirable by-products carbon monoxide and carbon dioxide are limited to a level that can be controlled by conventional technical means. On the one hand, this relates first of all to heat removal in the reactor due to the strong exotherm during the formation of carbon monoxide and carbon dioxide. As already mentioned, the oxygen is supplied in a purity of at least 95% by volume, so that the previously mentioned water vapor fraction in the feed mixture is the essential inert fraction in the feed mixture. The dilution of the essential reactive components ethane and oxygen is thus minimized compared to other systems known from literature. Optimum coordination of the catalyst properties and the process conditions is thus essential for the practical implementation of oxidative dehydrogenation under technical conditions, in order to ensure the desired requirements in terms of high value product yield and the reaction control and heat removal necessary for this. Equally, however, the effort required to remove carbon monoxide and carbon dioxide in the downstream decomposition section is reduced, and the portions of carbon monoxide and carbon dioxide formed can be removed with technically standard process steps. These include, for example, demethanization, in which the carbon monoxide is also removed, or catalytic conversion of the carbon monoxide and amine or caustic scrubbing for the removal of carbon dioxide.

The feed mixture is formed in the process according to the invention, in particular, in such a way that a ratio of the water vapor content in the feed mixture to a content of the at least one starting compound in the feed mixture is at least 0.23.

As mentioned, the invention is particularly suitable for use in connection with ODH, especially ODHE, so that in particular ethane is used as the feed compound and the oxidative catalytic process is carried out as oxidative dehydrogenation of ethane.

In particular, the invention provides for carrying out the oxidative catalytic process at a temperature of the catalyst or catalysts in a range between 240 and 500° C., more particularly between 280 and 450° C., further more particularly between 300 and 400° C., and/or carrying it out with a total pressure of the feed mixture at an inlet of the shell-and-tube reactor or reactors of 1 to 10 bar (abs.), more particularly 2 to 6 bar (abs.).

In the process according to the invention, the reaction tubes are cooled in particular using one or more cooling media flowing around the reaction tubes, as mentioned previously.

In embodiments of the invention, different sections of the reaction tubes may be cooled using different cooling media, using the same cooling medium in different cooling medium circuits, and/or using the same or different cooling media in different or the same flow directions.

In the context of the invention, the catalyst or catalysts are provided in different zones of the reaction tubes with different activities, as previously explained. In particular, a maximum temperature difference of 60 K, 55 K, 50 K, 45 K or 40 K is maintained between one or more temperature hotspots, in particular all temperature hotspots of the mentioned reaction zones, and a coolant temperature, for example a salt temperature. This leads to the avoidance of very high local temperatures. The maintenance of these maximum temperature differences is achieved by the mentioned provision of the catalysts with different activities, the activities are thus provided with their different activities in such a way that one or more of the temperature hotspots, in particular all of them, have the mentioned maximum temperature difference. A “temperature hotspot” is the position in the respective zone that has the highest temperature.

Advantageously, the catalyst or catalysts are made at least in part from the oxides of the metals.

The catalytically active material of the catalyst or catalysts can thus be produced from precursors that are commercially available in large quantities and at favorable prices. The disadvantages of production from (water-) soluble precursors of the metals, such as ammonium heptamolybdate or vanadyl sulfate, can be avoided in this way. Telluric oxide can be used instead of telluric acid. In particular, the catalytically active material can be prepared (completely) using the oxides also mentioned below.

The catalyst or catalysts is or are prepared in particular using a hydrothermal synthesis, in particular in an autoclave and in particular using a pressure in the range from 5 to 50 bar (abs.), in particular from 10 to 35 bar (abs.), further in particular from 15 to 30 bar (abs.), and a temperature in the range from 150 to 280° C., in particular from 150 to 230° C., further in particular from 170 to 210° C.

Further, the catalyst or catalysts is or are prepared in particular by crystallization under hydrothermal conditions using molybdenum trioxide, divanadium pentoxide, diniobium pentoxide and optionally tellurium dioxide and using oxo ligands, the oxo ligands in particular each having at least two oxygen atoms and being selected from carboxylic acids and alcohols, in particular from oxalic acid, citric acid and 1,2-alkanediols.

In particular, the catalyst or catalysts are formed into a particulate shape by compression in the context of the invention.

A catalytically active material, the actual catalyst, can be used in particular together with a catalytically inactive material which itself is not catalytically active but is provided together with the catalyst. The catalytically inactive material can be, for example, silica (SiO₂), aluminum oxide (AlO₂₃), silicon carbide (SiC) or graphite. In particular, silicon carbide and graphite are very advantageous inert materials for (strongly) exothermic reactions such as the oxidation of alkanes, especially ODH-E, since, in addition to the effect of dilution, they are particularly good thermal conductors and thus also contribute to effective thermal management of the reaction. For shaping (tabletting) of the catalysts, wax is also required, which is burned out after shaping and is therefore no longer present in the actual catalyst, but instead leaves behind corresponding pores that are important for the accessibility of the reactants to the catalytically active centers. The above-mentioned inert materials can be used for tabletting or as framework materials for suitable catalyst molded bodies of any type, or they can be further bodies not equipped with catalytically active material.

The catalyst or catalysts in particular have a catalytically inactive component, in particular a catalytically inactive metal oxide, and catalytic zones are advantageously provided in the reaction tubes in which the catalyst or catalysts is or are diluted in different amounts with the inactive metal oxide.

A plant for producing a target compound with one or more tube bundle reactors having reaction tubes arranged in parallel, which is adapted to form a feed mixture containing at least one reactant compound, to distribute it to reaction tubes of the tube bundle reactor(s), and to subject it to an oxidative catalytic reaction in the reaction tubes, is also an object of the invention.

This is set up to add water vapor to the feed mixture in an amount such that the water vapor content of the feed mixture is 5 to 95% by volume, and to add oxygen to the feed mixture in the form of a fluid containing at least 95% by volume oxygen, one or more catalysts containing the metals molybdenum, vanadium, niobium and optionally tellurium being used for the oxidative catalytic conversion in the reaction tubes.

In contrast to the above-mentioned established oxidation processes, the process according to the invention is based on the use of pure oxygen as oxidant. This pure oxygen can be easily and inexpensively provided from suitable sources, such as distillative air separation plants or even pressure swing adsorption.

The invention solves the problem of finding a catalyst adapted and optimized for the conditions used. As described, MoVNbOₓ and in particular MoVNbTeOₓ catalysts can be considered. According to the state of the art, the aim here is not only to provide as high a proportion of the so-called M1 phase as possible, but also to match the activity and selectivity precisely to the desired process conditions according to the invention.

Advantages and features of the invention and particularly preferred embodiments of the invention are summarized again below.

Typically, these catalysts are prepared by combining solutions of the soluble metal salts, such as ammonium heptamolybdate, vanadyl sulfate, telluric acid, and ammonium nioboxalate. The combined solution can be spray dried. Often, to increase the catalytically active phase (M1), another crystallization time under hydrothermal conditions (above 100° C. in water in an autoclave) is also added. This is followed by drying. However, the oxide catalyst is then formed during calcination under inert gas at over 550° C. These procedures are described in the literature, see, among others, A. Celaya Sanfiz et al, “Preparation of Phase-Pure M1 MoVTeNb Oxide Catalysts by Hydrothermal Synthesis-Influence of Reaction Parameters on Structure and Morphology,” Top. Catal. 50, 2008, 19-32 or D. Melzer et al, “Atomic-Scale Determination of Active Facets on the MoVTeNb Oxide M1 Phase and Their Intrinsic Catalytic Activity for Ethane Oxidative Dehydrogenation,” Angew. Chem. Int. Ed. 55, 2016, 8873-8877. Such a catalyst may also be part of the invention.

Surprisingly, the oxidative process used according to the invention, in particular an ODH(E) process, can be realized in a particularly advantageous manner with a less active catalyst compared to a catalyst typically used. As a rule, however, such a less active catalyst is economically disadvantageous, since lower yields are obtained or, for example, higher reaction temperatures have to be set, which increase the activity but in turn have a negative effect on the selectivity.

Furthermore, it was surprisingly found that a particularly suitable, i.e. less active, catalyst can be prepared if the catalyst synthesis is based on the corresponding metal oxides and not, as is usually the case, on soluble components and/or, where appropriate, tellurium dioxide. Such a catalyst can be prepared in particular by crystallization under hydrothermal conditions from the oxides of the metals using oxo-ligands, as specified in more detail below.

Surprisingly, a catalyst prepared in this way is significantly more selective than when other catalysts of the same composition are used. The oxo-ligands all have at least two oxygen atoms which can coordinate and the oxo-ligands are selected from the group of carboxylic acids and alcohols. In particular, the oxo ligand may be oxalic acid, citric acid and a 1,2-alkanediol such as (ethylene) glycol. Advantageously, the hydrothermal synthesis takes place in a closed autoclave in the range from 10 to 30 bar (abs.) and in the range from 150 to 230° C. (particularly preferably from 170 to 210° C.). A catalyst prepared in this way is described in DE 10 2017 000 861 A1. However, it was found there that the catalyst has a somewhat higher activity under other, anhydrous, very dilute process conditions than comparable catalysts from soluble precursors. A higher selectivity is not reported there. It is therefore all the more surprising that this catalyst exhibits lower activity but higher selectivity under the new process conditions according to the invention without inert gas and with water vapor in the reactant gas.

As a result, the use of such a catalyst under the process conditions according to the invention, in particular in the ODH(E), leads precisely to increased selectivity to the target product, i.e. in the ODH(E) to ethylene, since the activity is lower and thus a higher temperature is necessary, but at the same time a particularly high selectivity is achieved. Background principles are described, for example, in WO 2018/115416 A1, where the effect is used to adjust the product ratio of ethylene and acetic acid within certain limits as required by adjusting the feed gas composition and, in particular, the water content. In the present invention, however, the effect is used overall to selectively increase the proportion of value product (in the case of ODHE, in particular ethylene).

The guidance of the reaction in several reaction zones provided according to the invention, by catalyst layers of different activity gradations, has in particular the following advantages:
Increased operational safety due to lower risk of thermal runaway of the reaction.
Increased selectivity towards ethylene by maintaining a higher minimum temperature as well as a higher average temperature in the catalyst bed of the multiple reaction zones.
Increased conversion per run and associated with the increased selectivity to ethylene.
Increased ethylene yield by enabling operation at higher reactor/coolant temperatures due to increased operational safety/reduced risk of thermal runaway.

Within the scope of the invention, it is thus disclosed that a process for a selective oxidation of hydrocarbons can be carried out in a particularly advantageous manner by optimally combining aspects of reactor design, reaction control and catalyst preparation. In particular, such a selective oxidation of hydrocarbons may be an oxidative dehydrogenation (or oxyhydrogenation) of alkanes or alkenes having 1 to 6 carbon atoms. Preferably, such a process is an oxidative dehydrogenation of ethane.

### DESIGN EXAMPLES

The invention is further explained below with reference to examples corresponding to embodiments of the invention and comparative examples not in accordance with the invention, as well as associated figures and tables.

### Comparative Example

As an example of the commonly used synthesis of MoVNbOₓ and MoVNbTeOₓ catalysts, a catalyst designated “Catalyst 1” below was prepared starting from soluble starting compounds as described in Melzer et al. (see above, “Supplementary Material”).

### Example 1

As an example of a catalyst originating from tellurium dioxide, a catalyst prepared hereinafter as “Catalyst 2” was prepared based on DE 10 2017 000 848 A1, as described below.

In an autoclave (40 L), 3.3 L of distilled water were placed and heated to 80° C. with stirring. Meanwhile, 725.58 g of ammonium heptamolybdate tetrahydrate was added and dissolved (“AHM solution”). In two 5 L beakers, 1.65 L of distilled water was also heated to 80° C. in each beaker with stirring on a magnetic stirrer with temperature control. 405.10 g vanadyl sulfate hydrate (vanadium content 21.2%) and 185.59 g ammonium nioboxalate (niobium content 20.6%) were then added to each of these beakers and dissolved (“V solution” and “Nb solution”).

65.59 g of tellurium dioxide was ground in 200 g of distilled water for 3 h the previous day using a ball mill and transferred to a beaker with 1.45 L of distilled water (“Te suspension”).

Now, one after the other, the V solution was pumped into the AHM solution, then the Te suspension ground the day before was added, stirring continued for 1 h at 80° C., and finally the Nb solution was pumped into the AHM solution using a peristaltic pump. The resulting suspension was now stirred further for 10 min at 80° C., with the stirrer speed at 90 rpm during precipitation.

Subsequently, nitrogen was superimposed by building up a pressure of up to approx. 6 bar in the autoclave with nitrogen and opening the vent valve to such an extent that nitrogen flowed through the autoclave under pressure (5 min). At the end, the pressure was released again via the vent valve down to a residual pressure of 1 bar.

Hydrothermal synthesis in a 40-L autoclave was carried out at 175° C. for 20 h (heating time: 3 h) with an anchor stirrer at a stirrer speed of 90 rpm. After synthesis, filtration was performed using a vacuum pump with blue sand filter and the filter cake was washed with 5 liters of distilled water.

Drying was carried out at 80° C. in a drying oven for 3 days and then grinding was carried out in a beater mill, achieving a solid yield of 0.8 kg. Calcination was carried out at 280° C. for 4 h in an air stream (heating rate 5° C./min, 1 L/min air). Activation was carried out in a retort at 650° C. for 2 h in a nitrogen stream (heating rate 5° C./min, 0.5 L/min nitrogen).

### Example 2

As an example of a catalyst originating from the metal oxides, a catalyst hereinafter referred to as “Catalyst 3” was prepared based on DE 10 2017 000 861 A1, as described below.

Tellurium dioxide was slurried in 200 g of distilled water and ground in a planetary ball mill using 1 cm balls (zirconium dioxide). The portion was then transferred to a beaker with 500 ml of distilled water. Diniobpentoxide was slurried in 200 g of distilled water and ground in the same ball mill. The portion was then transferred to a beaker with 500 ml of distilled water. The next morning, the temperature was heated to 80° C., 107.8 g of oxalic acid dihydrate was added to the diniobpentoxide suspension, and stirred for about 1 h. The mixture was then removed from the suspension. In an autoclave (40 liters), 6 L of distilled water was placed and heated to 80° C. with stirring (stirrer speed 90 rpm).

When the water had reached the temperature, 61.58 g of citric acid, 19.9 g of ethylene glycol, 615.5 g of molybdenum trioxide, 124.5 g of divanadium pentoxide, the ground tellurium dioxide and the ground diniobpentoxide in oxalic acid were added successively. 850 ml of distilled water was used to transfer and rinse the vessels. The complete amount of water in the autoclave is 8.25 liters. Nitrogen was then added on top. Hydrothermal synthesis was performed in a 40-L autoclave at 190° C./48 hr. After synthesis, filtering was performed using a vacuum pump with blue sand filter and the filter cake was washed with 5 L of distilled water.

Drying was carried out at 80° C. in a drying oven for 3 days and then the product was ground in an impact mill. A solid yield of 0.8 kg was achieved.

Subsequent calcination was carried out at 280° C. for 4h in air (heating rate 5° C./min, 1 L/min air). Activation was carried out in a retort at 600° C. for 2h (heating rate 5° C./min, 0.5 L/min nitrogen).

### Example 3

The catalyst powders prepared as described previously were mixed with 2% graphite Timerex T44, 10% silica Siloid C809 powder and with 10% wax, compacted and then tableted into 3×3 mm tablets. These tablets were then split and a fraction of 1 to 2 mm was used as granules for the test. Afterwards, the wax was still burned out at 350° C. under air.

### Example 4

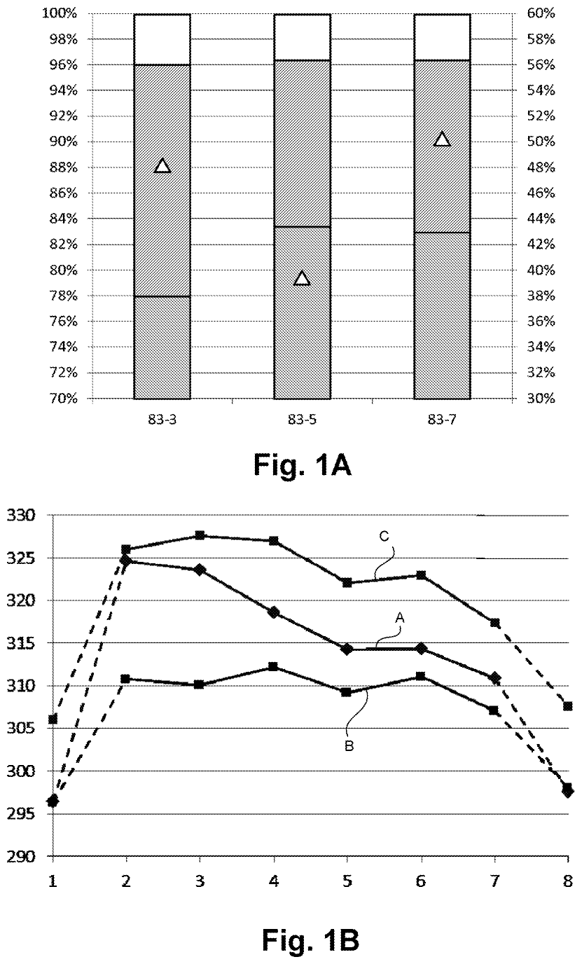

The catalysts produced in this way were investigated in an experimental plant with regard to their activity or their turnover-selectivity behavior. The reactor (usable length 0.9 m, inner diameter of the reaction chamber 10 mm) is designed as a double tube. Heating or cooling is performed by means of a thermal oil bath, where the thermal oil is pumped through the outer space of the reactor and thus heats the inner space/reaction zone or cools it at the same time (the conversion is an exothermic reaction). The exact test conditions are listed in Table 1. Results are shown in Table 2 and FIG. 1.

FIG. 1A illustrates the selectivities (left vertical axis; cross-hatching: ethylene, diagonal hatching: acetic acid, without filling: carbon oxides) and conversions (right vertical axis; triangles) of the catalysts according to the experimental points A, B and C shown in Table 1.

The following can be seen from the results:
Catalyst 3 based on the metal oxides shows an activity about 19% lower than catalyst 2 based on the soluble precursors and tellurium dioxide (see Table 2), i.e. lower ethane conversion at the same catalyst bed inlet temperature or the same coolant temperature
For the same conversion, catalyst 3 based on the pure oxides exhibits a selectivity to ethylene (and correspondingly 5% points lower selectivity to acetic acid) that is about 5% points higher than catalyst 2, with the same overall selectivity of more than 96% to the commercial value products ethylene and acetic acid, namely about 83% vs. about 78% selectivity to ethylene (catalyst 3 vs. catalyst 2) and approx. 13% vs. approx. 18% selectivity to acetic acid (catalyst 3 vs. catalyst 2), see FIG. 1, left.
Flatter temperature profile (even at higher catalyst bed inlet temperature) of catalyst 3 vs. catalyst 2 (FIG. 1 right) due to lower activity and lower selectivity to acetic acid (Ethane oxidation towards acetic acid is significantly more exothermic than ethane oxidation to ethylene; standard reaction enthalpy ethane to ethylene −105 kJ/mol, standard reaction enthalpy ethane to acetic acid −590 kJ/mol
Reduced risk of thermal runaway of the catalyst bed or part of the catalyst bed or a reaction zone in a commercial reactor.
A catalyst 3 prepared from the oxides shows a lower activity under these conditions, i.e. in particular with a lot of water vapor. To achieve the same conversion, a higher catalyst bed inlet temperature must be set due to this lower activity, which also requires a higher average or minimum catalyst bed temperature. Surprisingly, however, it now appears that catalyst 3 achieves a higher selectivity with the same conversion.
Surprisingly, therefore, the supposed disadvantage of a lower activity of a catalyst prepared via the pure oxides turns out to be particularly advantageous in the sense of the invention, since the reduced activity means that the process can/must be operated at somewhat elevated temperatures, which in the result then leads to an increased yield of particularly preferred value product ethylene.

TABLE 1

| | Test point | | |
|---|---|---|---|
| Conditions | A | B | C |
| Catalyst | Catalyst 2 | Catalyst 3 | |
| Catalyst mass [g] | 48.02 | | |
| Proportion of binder [wt. %] | 10 | | |
| Catalyst shape | Quartered 3 × 3 mm tablets | | |
| System pressure [bara] | 3.5 | | |
| WHSV [g_{C2H6}/(g_{cat} *h)] | 0.8 | | |
| Oil temperature [° C.] | 298 | 298 | 307.7 |
| Mean catalyst bed temperature [° C.] | 317.7 | 310.0 | 323.7 |
| O₂/C₂H₆ [mol/mol] | 0.373 | 0.373 | |
| H₂O/C₂H₆ [mol/mol] | 0.234 | 0.286 | |
| Feed composition [mol %] | | | |

| | | |
|---|---|---|
| C₂H₆ | 61.4 | 60.6 |
| O₂ | 24.2 | 22.0 |
| H₂O | 14.4 | 17.4 |

TABLE 2

test point

| | Cat. 2 | Cat. 3 |
|---|---|---|
| Conditions | (exp. point A) | (exp. point B) |
| Catalyst activity | 0.378 | 0.305 |
| [g_{C2H6} _{conversion}/(g_{cat} × h)] | | |
| Relative catalyst activity [%] | 100 | 81 |

There is a flatter temperature profile (even at higher catalyst bed inlet temperature) of catalyst 3 vs. catalyst 2 due to lower activity and lower selectivity to acetic acid. This effect can provide a reduction in the risk of thermal runaway of the catalyst bed or a portion of the catalyst bed or a reaction zone in a commercial reactor.

This circumstance is illustrated in FIG. 1B, in which the corresponding temperatures in ° C. on the vertical axis are plotted for measuring points before (measuring point 1) and after (measuring point 8) a catalyst bed of approx. 60 cm as well as measuring points (measuring points 2 to 7) within the catalyst bed on the horizontal axis.

### Example 5

A long-term test of a catalyst according to the invention was carried out in a pilot reactor. Using the optimized catalyst formulation described above corresponding to Catalyst 3, an appropriate amount of catalyst was prepared to fill a pilot-scale reactor.

The pilot reactor used is a fixed-bed reactor cooled with a molten salt. This is the same pilot reactor with which the results described in WO 2019/243480 A1 were obtained. The pilot reactor is designed as a tube-in-tube reactor, with the inner tube filled with the catalyst fixed bed (reaction chamber). Between the wall of the reaction chamber and the outer tube is the coolant chamber, i.e. this chamber is flowed through with the coolant, in this case a liquid molten salt, in countercurrent to the direction of flow of the reaction feed stream. The molten salt is a mixture of sodium nitrite, sodium nitrate and potassium nitrate. The dimensions (i.e., length, inner diameter, and wall thickness) of the pilot reactor reaction chamber are consistent with the typical dimensions of a single tube from a typical commercial (large-scale) shell-and-tube reactor. Thus, the pilot reactor can be regarded as a true replica of an industrial-scale plant (i.e., scale-up away from laboratory scale), since the same conditions (flow field, temperature or temperature gradients, pressure gradients, etc.) as in a technical shell-and-tube reactor are established in this pilot reactor due to its geometry, and thus the reaction can be tested under real technical conditions.

For the test operation, the pilot reactor was filled with a three-stage catalyst bed in terms of catalytic activity. The catalytically active base material was exactly the same for each stage. The bed was arranged in such a way that the catalytic activity increased in the flow direction of the reaction feed stream. The different activity gradation was achieved (as also described in WO 2019/243480 A1) by using catalyst molded bodies (rings) with different amounts of binder, which is needed to form the molded bodies, added to the exactly same catalytically active base material. Thus, the binder also acts as a diluent of the active catalyst material. Each catalyst layer had the same height and thus the same volume. Upstream and downstream of the three-stage catalyst bed was a bed of inert material of the same shape and size as the shaped catalyst bodies.

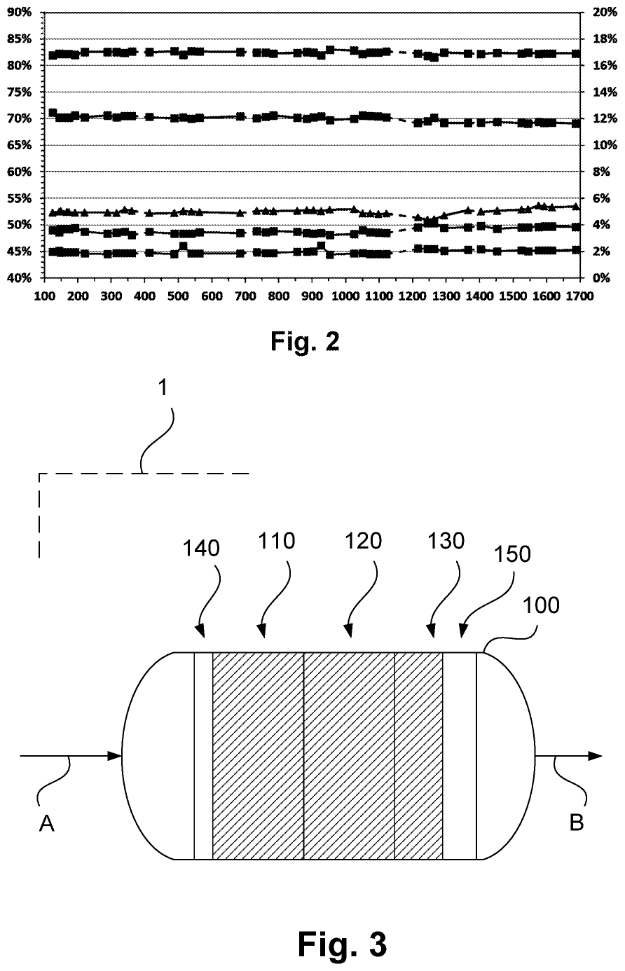

The pilot reactor was then operated for a period of about 1700 h (about 71 days) with a reaction feed stream consisting essentially of ethane, oxygen, and water (steam). The exact reaction conditions are listed in Table 3. Over the entire test period, a consistent, stable and very good reactor and catalyst performance was observed with respect to ethane conversion and selectivities to the desired commercial value products ethylene and acetic acid, as can also be seen in FIG. 2. The ethane conversion was about 52.5%, the selectivity to ethylene about 82.5% and the selectivity to acetic acid about 12%, i.e. a total selectivity to commercial value products of more than 94%.

TABLE 3

| Parameter | Unit | Value |
|---|---|---|
| Filled | Kg | 2.13 |
| catalyst active mass | | |
| Reactor inlet pressure | Bara | 3.81 |
| Medium | ° C. | 316 |
| coolant temperature | | |
| GHSV | h⁻¹ or (Nm³/h)_{gas}/m³_{cat.} | 1088 |

Composition reaction feed stream (molar ratio)
Ratio ethane:oxygen:water (vapor) = 59:24:17

The results of the stability tests are illustrated in FIG. 2, which shows an accumulated operating time (time on stream) in h on the horizontal axis versus a conversion of ethane on the left and a selectivity to ethylene on the right vertical axis, respectively, each in percent. From top to bottom, values are shown for selectivity to ethylene, selectivity to acetic acid, conversion of ethane, selectivity to carbon monoxide and selectivity to carbon dioxide.

### Example 6

FIG. 3 illustrates a plant for the production of olefins according to one embodiment of the invention in the form of a highly simplified plant diagram and is designated 1. Plant 1 is shown only schematically. In particular, the principle arrangement of the reaction zone(s) is illustrated by means of a greatly enlarged tubular reactor 100 which is not drawn to scale. Although a plant 1 for ODHE is described below, the invention is also suitable for use in ODH of higher hydrocarbons, as mentioned. In this case, the following explanations apply accordingly.

As mentioned, the plant 1 has a shell-and-tube reactor 100 to which, in the example shown, a feed mixture A containing ethane and obtained in any manner is fed. The feed mixture A may contain, for example, hydrocarbons taken from a rectification unit not shown. The feed mixture A may also be, for example, preheated and otherwise processed. The feed mixture A may already include oxygen and, optionally, a reaction moderator such as steam, but corresponding media may also be added upstream or in the shell-and-tube reactor 100, as not shown separately. A product mixture B is removed from the tubular reactor 100.

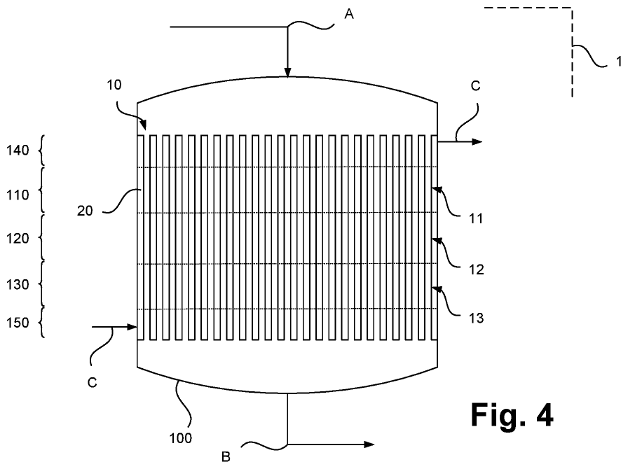

The reactor 100, shown in detail in FIG. 4, has a plurality of parallel reaction tubes 10 (only partially labeled) extending through a preheating zone 140 and then through a plurality of reaction zones 110, 120, 130, three in the example shown. Downstream, a post-reaction zone 150 may be present. The reaction tubes 10 are surrounded by a jacket region 20 through which, in the example, a coolant C of the type explained is passed. The embodiment is greatly simplified because, as mentioned, the reaction tubes 10 may be cooled using multiple cooling media flowing around the reaction tubes 10, or different tube sections may be cooled using different cooling media, the same cooling media in different cooling media circuits, and/or the same or different cooling media in different or the same flow directions.

After being fed into the shell-and-tube reactor, the feed mixture A is suitably distributed to the reaction tubes 10 at a temperature in a first temperature range. The reaction tubes have respective catalytic zones 11, 12 and 13 located in the reaction zones 120, 130 and 140.

A catalytic conversion is carried out by means of the catalytic zones 11, 12 and 13 arranged one behind the other in the reaction tubes 10, which can optionally be provided and in the process can, for example, have a different activity and/or selectivity. The parameters of the feed mixture and the reaction conditions have been explained several times.

1. A method for producing a target compound, comprising:
forming a feed mixture containing at least one reactant compound;
distributing the feed mixture to parallel reaction tubes of one or more shell-and-tube reactors; and
subjecting the feed mixture to an oxidative catalytic reaction in the reaction tubes, wherein ethane is used as reactant compound and the oxidative catalytic process is carried out as oxidative dehydrogenation of the ethane;
wherein:
steam is added to the feed mixture in an amount such that a steam content of the feed mixture is from 5 to 95% by volume;
oxygen is added to the feed mixture in the form of a fluid containing at least 95% by volume of oxygen; and
the oxidative catalytic reaction is carried out using one or more catalysts containing the metals molybdenum, vanadium, and niobium which is or are at least partially made from the oxides and which is or are arranged in a plurality of reaction zones of the one or more shell-and-tube reactors, wherein the one or the plurality of catalysts in the reaction zones is or are provided with a different catalyst loading and/or a different catalyst activity per unit space.

2. (canceled)

3. The method according to claim 1, wherein the water vapor content of the feed mixture is 10 to 50% by volume.

4. The method according to claim 1, wherein the feed mixture is formed such that, in the feed mixture, a ratio of a proportion of oxygen to a proportion of the at least one reactant compound is at least 0.20 and up to 1.0.

5. The method according to claim 1, wherein the feed mixture is formed such that a ratio of the amount of water vapor in the feed mixture to an amount of the at least one reactant compound in the feed mixture is at least 0.23.

6. (canceled)

7. The method according to claim 1, wherein the oxidative catalytic process is carried out at a temperature of the catalyst or catalysts in a range between 240 and 500° C., and/or in which a total pressure of the feed mixture at an inlet of the shell-and-tube reactor or reactors is 1 to 10 bar (abs.).

8. The method according to claim 1, wherein the reaction tubes are cooled using one or more cooling media flowing around the reaction tubes.

9. The method according to claim 8, wherein different sections of the reaction tubes are cooled using different cooling media, using the same cooling medium in different cooling medium circuits, and/or using the same or different cooling media in different or the same flow directions.

10. The method according to claim 1, wherein a maximum temperature difference of 60 K is maintained between one or more temperature hotspots of the reaction zones and a coolant temperature.

11. The method according to claim 1, wherein the catalyst or catalysts is or are prepared using a hydrothermal synthesis, in particular in an autoclave and in particular using a pressure in the range from 5 to 50 bar (abs.) and a temperature in the range from 150 to 280° C.

12. The method according to claim 11, wherein the catalyst or catalysts are prepared by crystallization under hydrothermal conditions using molybdenum trioxide, divanadium pentoxide and using oxo ligands, the oxo ligands in particular each having at least two oxygen atoms and being selected from carboxylic acids and alcohols, in particular from oxalic acid, citric acid and 1,2-alkanediols.

13. The method according to claim 12, wherein the catalyst or catalysts is or are formed into a particulate shape by compression.

14. The method according to claim 13, wherein the catalyst or catalysts in particular comprise a catalytically inactive component, in particular a catalytically inactive metal oxide, and wherein catalytic zones are provided in the reaction tubes in which the catalyst or catalysts are diluted in different amounts with the inactive metal oxide.

15. A plant for producing a target compound, comprising:
one or more tube shell-and-tube reactors having reaction tubes arranged in parallel, the one or more reactors being adapted to:
form a feed mixture containing at least one reactant compound;
distribute the feed mixture to reaction tubes of the tube bundle reactor(s); and
subject the feed mixture to an oxidative catalytic reaction in the reaction tubes;
wherein:
the plant is adapted to add steam to the feed mixture in an amount such that a steam fraction of the feed mixture is 5 to 95% by volume; and
oxygen is added to the feed mixture in the form of a fluid containing at least 95% by volume oxygen; and
one or more catalysts containing the metals molybdenum, vanadium, and niobium are used for the oxidative catalytic reaction in the reaction tubes.

16. The plant according to claim 15, wherein the catalyst further contains tellurium.

17. The method according to claim 1, wherein the catalyst further contains tellurium.

18. The method according to claim 3, wherein the water vapor content of the feed mixture is 14 to 35% by volume.

19. The method according to claim 2, wherein the water vapor content of the feed mixture is 10 to 50% by volume.

20. The method according to claim 7, wherein the oxidative catalytic process is carried out at a temperature of the catalyst or catalysts in a range between 300 and 400° C.-and/or in which a total pressure of the feed mixture at an inlet of the shell-and-tube reactor or reactors is 2 to 6 bar (abs.).
